(19) United States
(12) Patent Application Publication
(10) Pub. No.: US 2026/0325796 A1
(43) Pub. Date: Oct. 22, 2026

(54) Catheter Tips for Rapidly Insertable Central Catheters and Methods Thereof

(71) Applicant: Bard Access Systems, Inc., Salt Lake City, UT (US)

(72) Inventor: Chase Provost, West Bountiful, UT (US)

(21) Appl. No.: 19/184,914

(22) Filed: Apr. 21, 2025

(51) Int. Cl. A61M 25/06; A61M 25/00

(52) U.S. Cl. CPC A61M 25/06; A61M 25/0014; A61M 25/0097

(57) ABSTRACT

Rapidly insertable central catheters (“RICCs”) and methods thereof allow clinicians to skip dilation of tissue with separate dilators when placing the foregoing RICCs. A RICC can include, a catheter tube, a single-piece catheter tip coupled to a distal end portion of the catheter tube, a catheter hub coupled to a proximal portion of the catheter tube, and one or more extension legs, wherein each extension leg of the one or more extension legs is coupled to the catheter hub by a distal portion thereof. The catheter tube can include a catheter-tube portion of a primary lumen of the RICC. The catheter tip can include a beveled section including a bevel approximating an obliquely truncated cone, a main section proximal of the beveled section, and a catheter-tip portion of the primary lumen of the RICC.

### BACKGROUND

An acute central venous catheter (“ACVC”) is typically inserted using the Seldinger technique. In accordance with the Seldinger technique, a needle tract to a blood-vessel lumen is dilated with a separate dilator prior to advancing the ACVC over an access guidewire into the blood-vessel lumen for subsequent placement. It would be advantageous if clinicians could skip dilation of the blood-vessel lumen with the separate dilator. However, current ACVCs have relatively blunt catheter tips making it difficult to do so.

Disclosed herein are catheter tips for rapidly insertable central catheter (“RICCs”) and methods thereof that address the foregoing.

### SUMMARY

Disclosed herein is a RICC including, in some embodiments, a catheter tube, a single-piece catheter tip coupled to a distal end portion of the catheter tube, a catheter hub coupled to a proximal end portion of the catheter tube, and one or more extension legs, wherein each extension leg of the one or more extension legs is coupled to the catheter hub by a distal end portion thereof. The catheter tube includes a catheter-tube portion of a primary lumen of the RICC. The catheter tip includes a beveled section including a bevel approximating an obliquely truncated cone, a main section proximal of the beveled section, and a catheter-tip portion of the primary lumen of the RICC.

In some embodiments, the beveled section is configured for dilating tissue around a needle tract from a size commensurate with an outer diameter of a needle shaft of an introducer needle to a size commensurate with an outer diameter of a distalmost portion of the main section of the catheter tip.

In some embodiments, the catheter-tip portion of the primary lumen of the RICC is offset with respect to a center of a transverse cross section of the main section of the catheter tip.

In some embodiments, the bevel is coaxial with respect to the catheter-tip portion of the primary lumen of the RICC. The bevel has a circular perimeter around its distal opening concentric with an inner diameter of the catheter-tip portion of the primary lumen of the RICC.

In some embodiments, the bevel has an elliptical base oblique to its distal opening.

In some embodiments, the main section of the catheter tip includes a straight portion and a tapered portion proximal of the straight portion. The straight portion of the main section has a consistent outer diameter. The tapered portion of the main section has a non-uniform taper configured for dilating tissue around a needle tract from a size commensurate with an outer diameter of a proximalmost portion of the beveled section to a size commensurate with an outer diameter of the distal end of the catheter tube.

In some embodiments, the bevel is coaxial with respect to the straight portion of the main section of the catheter tip. The bevel has a circular perimeter around its base concentric with the outer diameter of the straight portion of the main section.

In some embodiments, the bevel has an elliptical distal opening oblique to its base.

In some embodiments, each successive transverse cross section of the tapered portion of the main section of the catheter tip approximates a larger eccentric annulus in a proximal direction and a smaller eccentric annulus in a distal direction along the tapered portion of the main section. Each eccentric annulus of the foregoing larger and smaller eccentric annuli is defined by an outer diameter of the tapered portion of the main section and an inner diameter of the catheter-tube portion of the primary lumen of the RICC, thereby providing the non-uniform taper of the tapered portion of the main section of the catheter tip.

In some embodiments, the catheter tip is of a first polymeric material and the catheter tube is of a second, softer polymeric material.

In some embodiments, the first polymeric material is sufficiently stiff to prevent the catheter tip from collapsing, buckling, or otherwise appreciably deforming when a distal portion of the RICC is advanced into a blood-vessel lumen of a patient. The first polymeric material is also sufficiently pliable to prevent trauma to the blood-vessel lumen when the distal portion of the RICC is advanced farther into the blood-vessel lumen.

In some embodiments, the RICC includes a set of three lumens including the primary lumen, a secondary lumen, and a tertiary lumen. The secondary and tertiary lumens terminate in the distal end portion of the catheter tube by at least some infill of melted polymeric material of the first polymeric material.

In some embodiments, the primary lumen has a primary-lumen aperture in a distal opening of the catheter tip. The secondary lumen has a secondary-lumen aperture in a side of the distal end portion of the catheter tube. The tertiary lumen has a tertiary-lumen aperture in the side of the distal end portion of the catheter tube but proximal of the secondary-lumen aperture.

Also disclosed herein is a catheter tip including, in some embodiments, a beveled section including a bevel approximating an obliquely truncated cone, a main section proximal of the beveled section, a lumen through an entirety of the catheter tip.

In some embodiments, the lumen is offset with respect to a center of a transverse cross section of the main section of the catheter tip.

In some embodiments, the bevel is coaxial with respect to the lumen. The bevel has a circular perimeter around its distal opening concentric with an inner diameter of the lumen.

In some embodiments, the bevel has an elliptical base oblique to its distal opening.

In some embodiments, the main section of the catheter tip includes a straight portion and a tapered portion proximal of the straight portion. The straight portion of the main section has a consistent outer diameter. The tapered portion of the main section has a non-uniform taper.

In some embodiments, the bevel is coaxial with respect to the straight portion of the main section of the catheter tip. The bevel has a circular perimeter around its base concentric with the outer diameter of the straight portion of the main section.

In some embodiments, the bevel has an elliptical distal opening oblique to its base.

In some embodiments, each successive transverse cross section of the tapered portion of the main section of the catheter tip approximates a larger eccentric annulus in a proximal direction and a smaller eccentric annulus in a distal direction along the tapered portion of the main section. Each eccentric annulus of the foregoing larger and smaller eccentric annuli is defined by an outer diameter of the tapered portion of the main section and an inner diameter of the lumen, thereby providing the non-uniform taper of the tapered portion of the main section of the catheter tip.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates a RICC in accordance with some embodiments.

FIG. 2 illustrates a detailed view of a catheter tip coupled to a distal end portion of a catheter tube in accordance with some embodiments.

FIG. 3 illustrates a longitudinal cross section of the catheter tip and catheter tube of FIG. 2 in accordance with some embodiments.

FIG. 4A illustrates a detailed view of the catheter tip in accordance with some embodiments.

FIG. 4B illustrates a detailed view of the catheter tip in accordance with some other embodiments.

FIG. 5 illustrates obliquely truncated cones in accordance with some embodiments.

FIG. 6 illustrates a transverse cross section of the distal end portion of the catheter tube in accordance with some embodiments.

FIG. 7 illustrates transverse cross sections of a tapered portion of the catheter tip in accordance with some embodiments.

FIG. 8 illustrates a method for forming the catheter tip coupled to the catheter tube in accordance with some embodiments.

FIG. 9 illustrates a bevel-cutting step of the method for forming the catheter tip in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. “Proximal” refers to a portion, section, piece, element, or the like of a medical device intended to be near or relatively nearer to a clinician when the medical device is used on a patient. For example, a “proximal portion” or “proximal section” of the medical device includes a portion or section of the medical device intended to be near the clinician when the medical device is used on the patient. Likewise, a “proximal length” of the medical device includes a length of the medical device intended to be near the clinician when the medical device is used on the patient. A “proximal end” of the medical device is an end of the medical device intended to be near the clinician when the medical device is used on the patient. The proximal portion, the proximal section, or the proximal length of the medical device need not include the proximal end of the medical device. Indeed, the proximal portion, the proximal section, or the proximal length of the medical device can be short of the proximal end of the medical device. However, the proximal portion, the proximal section, or the proximal length of the medical device can include the proximal end of the medical device. Should context not suggest the proximal portion, the proximal section, or the proximal length of the medical device includes the proximal end of the medical device, or if it is deemed expedient in the following description, “proximal portion,” “proximal section,” or “proximal length” can be modified to indicate such a portion, section, or length includes an end portion, an end section, or an end length of the medical device for a “proximal end portion,” a “proximal end section,” or a “proximal end length” of the medical device, respectively.

“Distal” refers to a portion, section, piece, element, or the like of a medical device intended to be near, relatively nearer, or even in a patient when the medical device is used on the patient. For example, a “distal portion” or “distal section” of the medical device includes a portion or section of the medical device intended to be near, relatively nearer, or even in the patient when the medical device is used on the patient. Likewise, a “distal length” of the medical device includes a length of the medical device intended to be near, relatively nearer, or even in the patient when the medical device is used on the patient. A “distal end” of the medical device is an end of the medical device intended to be near, relatively nearer, or even in the patient when the medical device is used on the patient. The distal portion, the distal section, or the distal length of the medical device need not include the distal end of the medical device. Indeed, the distal portion, the distal section, or the distal length of the medical device can be short of the distal end of the medical device. However, the distal portion, the distal section, or the distal length of the medical device can include the distal end of the medical device. Should context not suggest the distal portion, the distal section, or the distal length of the medical device includes the distal end of the medical device, or if it is deemed expedient in the following description, “distal portion,” “distal section,” or “distal length” can be modified to indicate such a portion, section, or length includes an end portion, an end section, or an end length of the medical device for a “distal end portion,” a “distal end section,” or a “distal end length” of the medical device, respectively.

Unless defined otherwise, all technical and scientific terms used herein have the same meaning as commonly understood by those of ordinary skill in the art.

Again, an ACVC is typically inserted using the Seldinger technique. In accordance with the Seldinger technique, a needle tract to a blood-vessel lumen is dilated with a separate dilator prior to advancing the ACVC over an access guidewire into the blood-vessel lumen for subsequent placement. It would be advantageous if clinicians could skip dilation of the blood-vessel lumen with the separate dilator. However, current ACVCs have relatively blunt catheter tips making it difficult to do so.

Disclosed herein are catheter tips for RICCs and methods thereof that address the foregoing.

### RICCs

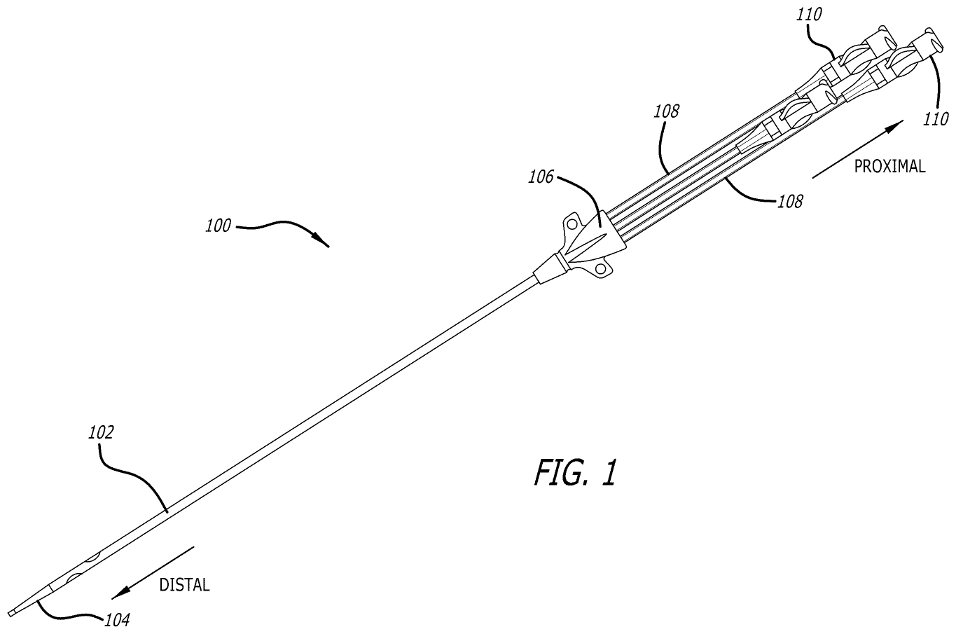

FIG. 1 illustrates a RICC 100 in accordance with some embodiments.

As shown, the RICC 100 can include a catheter tube 102 having a single-piece catheter tip 104 coupled thereto, a catheter hub 106, and one or more extension legs 108. The catheter tip 104 can be coupled to a distal end portion of the catheter tube 102, and the catheter hub 106 can be coupled to a proximal end portion of the catheter tube 102. Each extension leg of the one or more extension legs 108 can be coupled to the catheter hub 106 by a distal end portion thereof. In addition, one or more extension-leg connectors 110 (e.g., Luer connectors) can be coupled to the one or more extension legs 108 for connecting one or more other medical devices, respectively.

The RICC 100 can be a monoluminal RICC or a multiluminal RICC (e.g., a diluminal RICC, a triluminal RICC, a tetraluminal RICC, a pentaluminal RICC, a hexaluminal RICC, etc.). For example, the RICC 100 can be a triluminal RICC including a set of three lumens as provided in FIG. 1. (See also FIG. 6.) The set of three lumens can include a primary lumen 112 having an inner diameter (e.g., any of d_{Ai}, d_{Bi}, or d_{Ci} of FIG. 7), a secondary lumen 114, and a tertiary lumen 116. Each lumen of the primary lumen 112, the secondary lumen 114, and the tertiary lumen 116 of the RICC 100 can be formed of at least three fluidly connected luminal portions, namely those of a catheter-tube lumen, a catheter-hub lumen, and an extension-leg lumen of three catheter-tube lumens 118, 120, and 122, three catheter-hub lumens, and three extension-leg lumens in the RICC 100. For instance, the primary lumen 112 of the RICC 100 can be formed of a primary catheter-tube lumen 118, a primary catheter-hub lumen, and a primary extension-leg lumen; however, the primary lumen 112 of the RICC 100 can further include a fluidly connected catheter-tip lumen 124 of the catheter tip 104 as well. Further, the secondary lumen 114 of the RICC 100 can be formed of a secondary catheter-tube lumen 120, a secondary catheter-hub lumen, and a secondary extension-leg lumen. Lastly, the tertiary lumen 116 of the RICC 100 can be formed of a tertiary catheter-tube lumen 122, a tertiary catheter-hub lumen, and a tertiary extension-leg lumen. The primary lumen 112 can have a primary-lumen aperture 126 corresponding to a distal opening in a distal end of the catheter tip 104, which corresponds to a distal end of the RICC 100. The secondary lumen 114 can have a secondary-lumen aperture 128 in a side of a distal portion or, more specifically, the distal end portion of the catheter tube 102. The tertiary lumen 116 can have a tertiary-lumen aperture 130 in the side of the distal portion or, more specifically, the distal end portion of the catheter tube 102 proximal of the secondary-lumen aperture 128. Like the secondary and tertiary lumens 114 and 116, each additional lumen can have a corresponding lumen aperture in the side of the foregoing distal portion of the catheter tube 102 proximal of its predecessor.

### Catheter Tips

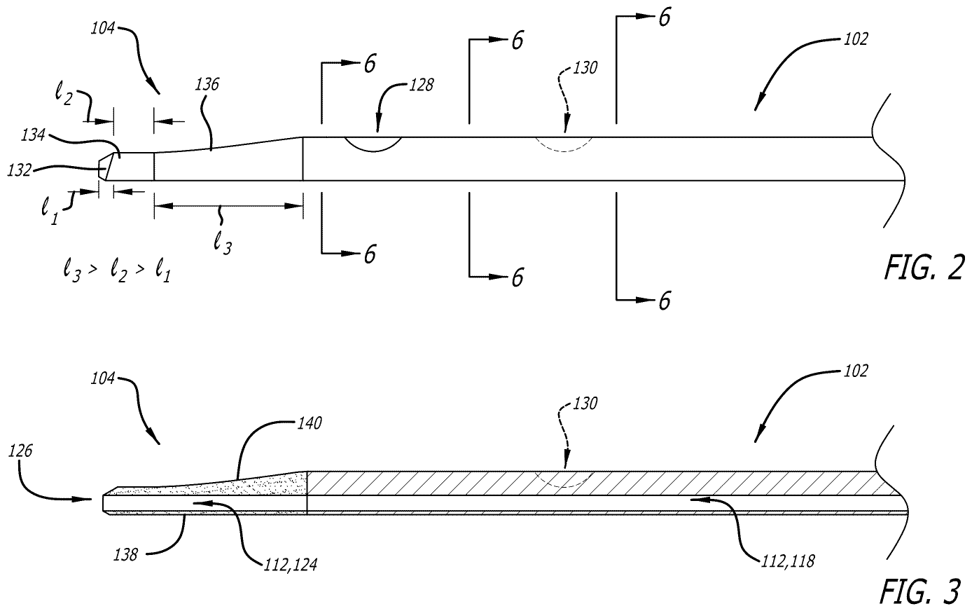

FIG. 2 illustrates a detailed view of the catheter tube 102 with the catheter tip 104 coupled thereto in accordance with some embodiments. FIG. 3 illustrates a longitudinal cross section of the catheter tube 102 with the catheter tip 104 in accordance with some embodiments. FIGS. 6 and 7 illustrate cross sections of the catheter tube 102 and the catheter tip 104 in accordance with some embodiments.

Beginning with the catheter tube 102 to which the catheter tip 104 is coupled, the catheter tube 102 can include one or more catheter-tube lumens such as the three catheter-tube lumens 118, 120, and 122 set forth above. Indeed, when the RICC 100 is a triluminal RICC, the catheter tube 102 can include a catheter-tube portion of the primary lumen 112 of the RICC 100, a catheter-tube portion of the secondary lumen 114 of the RICC 100, and a catheter-tube portion of the tertiary lumen 116 of the RICC 100. Before forming the catheter tip 104 coupled to the catheter tube 102 as set forth in the method below, each catheter-tube lumen of the foregoing three catheter-tube lumens 118, 120, and 122 can extend completely through the catheter tube 102. Subsequent to forming the catheter tip 104, however, only the primary catheter-tube lumen 118 typically extends from a proximal end of the catheter tube 102 to a distal end of the catheter tube 102. Indeed, the primary catheter-tube lumen 118 typically extends through both the proximal and distal ends of the catheter tube 102 and continues to extend through the catheter-tip lumen 124 of the catheter tip 104 where it is notably offset with respect to a center of a transverse cross section of the main section of the catheter tip 104. That is, a centerline through the catheter-tip lumen 124 is offset with respect to the center of a transverse cross section of the main section of the catheter tip 104. In contrast to extension of the primary catheter-tube lumen 118 through the catheter tip 104 by way of the catheter-tip lumen 124, the secondary and tertiary catheter-tube lumens 120 and 122 typically terminate in the distal end portion of the catheter tube 102 by at least some infill of the melted polymeric material of the first polymeric material used to form the catheter tip 104.

The catheter tube 102 can be formed of a second, softer polymeric material than the first polymeric material of the catheter tip 104 set forth below. In other words, the second polymeric material can have a second durometer less than the first durometer of the first polymeric material. Such a second polymeric material can include, but is not limited to, a polyvinyl chloride, a polyethylene, a polyurethane, or a silicone having the second durometer less than the first durometer of the first polymeric material. For example, if the catheter tip 104 is formed of a polyurethane as set forth below, the catheter tube 102 can be formed of a different polyurethane (e.g., a same or different diisocyanate or triisocyanate reacted with a different diol or triol, a different diisocyanate or triisocyanate reacted with a same or different diol or triol, a same diisocyanate or triisocyanate reacted with a same diol or triol under different conditions or with different additives, etc.) having a second durometer less than a first durometer of the polyurethane of the catheter tip 104. Notably, polyurethanes can be advantageous for the catheter tube 102 in that polyurethanes are relatively stiff at room-temperature but become more pliable in vivo at body temperature, which reduces irritation to blood-vessel walls as well as phlebitis. Polyurethanes are also advantageous in that they can be less thrombogenic than some other polymers. Whether or not the second polymeric material is a polyurethane, the second polymeric material can be configured to be sufficiently stiff ex vivo to impart a column strength to the catheter tube 102 that prevents the catheter tube 102 from collapsing, buckling, or otherwise appreciably deforming when the RICC 100 is advanced into the blood-vessel lumen per the method of placing the RICC 100 set forth below.

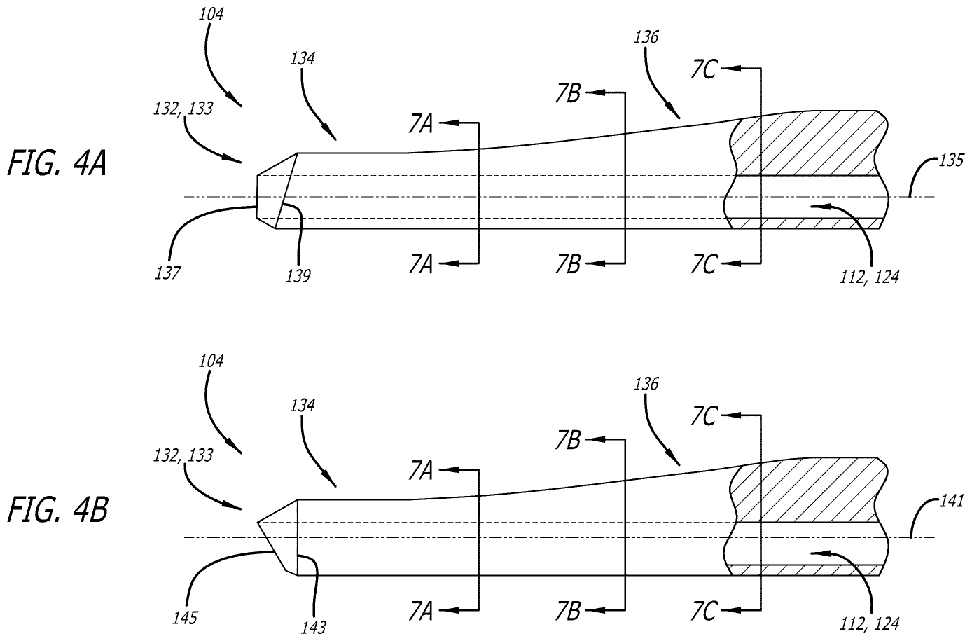

FIG. 2 illustrates a detailed view of the catheter tip 104 coupled to the catheter tube 102 in accordance with some embodiments. FIG. 3 illustrates a longitudinal cross section of the catheter tip 104 coupled to the catheter tube 102 in accordance with some embodiments. FIGS. 4A and 4B illustrate detailed views of the catheter tip 104 in accordance with some embodiments. FIG. 7 illustrates transverse cross sections of the tapered portion 136 of the catheter tip 104 in accordance with some embodiments.

As shown, the catheter tip 104 can have a beveled section 132 and a main section proximal of the beveled section 132, which main section includes a straight portion 134 and a tapered portion 136 proximal of the straight portion 134. The straight portion 134 of the catheter tip 104 can have a length l₂ longer than a length l₁ of the beveled section 132 of the catheter tip 104, and the tapered portion 136 of the catheter tip 104 can have a length l₃ longer than the length l₂ of the straight portion 134 of the catheter tip 104. For example, the length l₂ of the straight portion 134 of the catheter tip 104 can be about 4.0 to 5.0 times longer than the length l₁ of the beveled section 132 of the catheter tip 104, and the length l₃ of the tapered portion 136 of the catheter tip 104 can be about 4.5 to 7.5 times longer than the length l₂ of the straight portion 134 of the catheter tip 104. However, it should be understood relative lengths disclosed herein such as those given for the beveled section 132 and the main section of the catheter tip 104, as well as dimensions disclosed herein, generally relate to a 6-8 Fr RICC such as a 7 Fr RICC. The relative lengths and dimensions for larger RICCS (e.g., 7-14 Fr) or smaller RICCs (e.g., 4-6 Fr) as measured on the French catheter scale can vary, optionally, proportionally, by which the relative lengths and dimensions for the larger RICCs and smaller RICCs are also disclosed.

Beginning with the beveled section 132 of the catheter tip 104, the beveled section 132 can include a bevel 133 configured for immediately dilating tissue around a needle tract formed with an introducer needle as the RICC 100 is advanced into a blood-vessel lumen of a patient per the method of placing the RICC 100 set forth below. The beveled section 132 of the catheter tip 104 can dilate the tissue around the needle tract from a size commensurate with an outer diameter of a needle shaft of the introducer needle to a size commensurate with an outer diameter of a distalmost portion of the main section of the catheter tip 104, specifically, the outer diameter of the straight portion 134 of the catheter tip 104, at its least. Being that the beveled section 132 of the catheter tube 102 is not blunt like a catheter tip of a typical ACVC, the beveled section 132 of the catheter tip 104 can reduce the force needed to insert the RICC 100 into the needle tract over that of the typical ACVC.

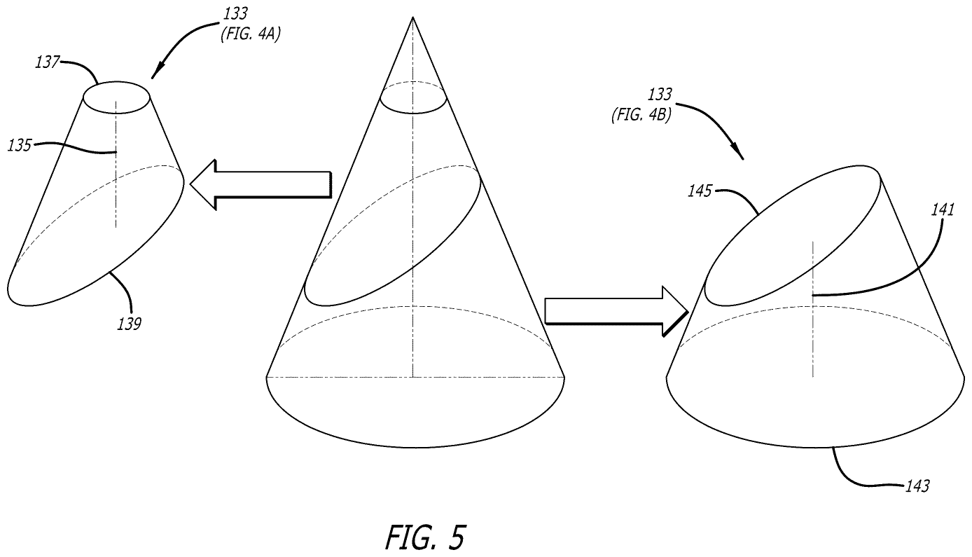

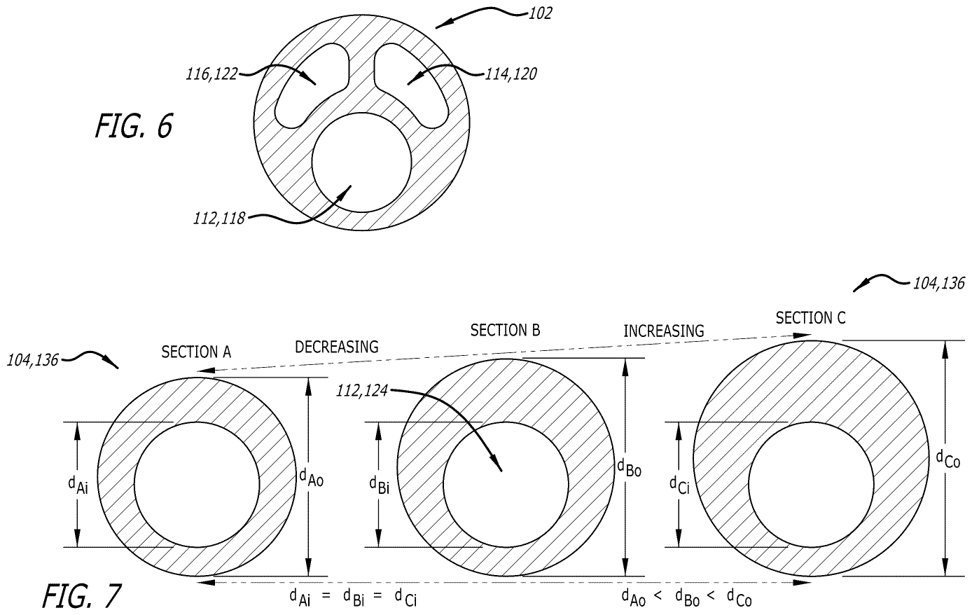

FIGS. 4A and 4B illustrate detailed views of the bevel 133 of the beveled section 132 of the catheter tip 104 in accordance with some embodiments. FIG. 5 illustrates obliquely truncated cones approximated by the bevel 133 in accordance with some embodiments.

As shown, the bevel 133 of the beveled section 132 of the catheter tip 104 can approximate an obliquely truncated cone. More specifically, an abluminal surface of the bevel 133 approximates a surface of an obliquely truncated cone.

Indeed, the bevel 133 of the catheter tip 104 shown in FIG. 4A can be considered coaxial with respect to the catheter-tip lumen 124 of the catheter tip 104 or, in the case of a multiluminal RICC, the catheter-tip portion of the primary lumen 112 of the RICC 100 in that a hypothetical cone from which the bevel 133 is excised is coaxial with respect to a central axis 135 defined by the catheter-tip lumen 124. Such a bevel 133 has a circular perimeter around its distal lip 137 concentric with an inner diameter of the catheter-tip lumen 124. Should the bevel 133 have a negligible lip around the distal lip 137, the circular perimeter around the distal lip 137 is also commensurate with the inner diameter of the catheter-tip lumen 124. Due to the catheter-tip lumen 124 being offset with respect to the center of a transverse cross section of the main section of the catheter tip 104, the bevel 133 has an elliptical base 139 oblique to its distal lip 137.

The bevel 133 of the catheter tip 104 shown in FIG. 4B can be considered coaxial with respect to the straight portion 134 of the catheter tip 104 in that a hypothetical cone from which the bevel 133 is excised is coaxial with respect to a central axis 141 defined by the outer diameter of the straight portion 134 of the catheter tip 104. Such a bevel 133 has a circular perimeter around its base 143 concentric and commensurate with the outer diameter of the straight portion 134 of the catheter tip 104. Further to the catheter-tip lumen 124 being offset with respect to the center of a transverse cross section of the main section of the catheter tip 104, the bevel 133 has an elliptical distal lip 145 oblique to its base 143.

Continuing with the main section of the catheter tip 104, the straight portion 134 can have a relatively consistent outer diameter along the straight portion 134 as shown in FIGS. 4A and 4B. In contrast, the tapered portion 136 can have a non-uniform taper having a variable taper angle over an outer diameter thereof as shown in FIGS. 4A, 4B, and 7. (See FIG. 7, wherein the outer diameter of the tapered portion 136 of the catheter tip 104 is indicated by d_{Ao}, d_{Bo}, and d_{Co}.) Such a non-uniform taper can be configured for further dilating the tissue around the needle tract as the RICC 100 is advanced into the blood-vessel lumen of the patient per the method of placing the RICC 100 set forth below. The non-uniform taper of the tapered portion 136 of the catheter tip 104 can dilate the tissue around the needle tract from a size commensurate with a proximalmost portion of the beveled section 132 or the outer diameter of the straight portion 134 of the catheter tip 104 to a size commensurate with an outer diameter of the catheter tube 102. Further being that the non-uniform taper of the tapered portion 136 of the catheter tube 102 is not blunt like the catheter tip of the typical ACVC, the tapered portion 136 of the catheter tip 104 can further reduce the force needed to insert the RICC 100 into the needle tract over that of the typical ACVC. Indeed, the tapered portion 136 of the catheter tip 104 is configured to integrate into the catheter tip 104 dilation normally done by a dilator in the Seldinger technique, thereby reducing the force needed to insert the RICC 100 into the needle tract and eliminating a separate dilation step.

As shown in FIG. 7, each successive transverse cross section of the tapered portion 136 of the catheter tip 104 can approximate a larger eccentric annulus in a proximal direction along the tapered portion 136 of the catheter tip 104. In addition, each successive transverse cross section of the tapered portion 136 of the catheter tip 104 can approximate a smaller eccentric annulus in a distal direction along the tapered portion 136 of the catheter tip 104. Each eccentric annulus of the foregoing larger and smaller eccentric annuli can be defined by the outer diameter (e.g., d_{Ao}, d_{Bo}, or d_{Co}) of the tapered portion 136 of the catheter tip 104 and the inner diameter (e.g., d_{Ai}, d_{Bi}, or d_{Ci}) of the catheter-tube portion of the primary lumen 112 of the RICC 100, thereby providing the non-uniform taper of the tapered portion 136 of the catheter tip 104.

Notably, the taper angle of the non-uniform taper of the tapered portion 136 of the catheter tip 104 can vary in the following way to provide the larger and smaller eccentric annuli of each successive transverse cross section of the tapered portion 136. Consider, for example, an abluminal surface of the tapered portion 136 of the catheter tip 104 ruled such that each point on the abluminal surface is on a longitudinal surface line of a plurality of circumferentially spaced surface lines that includes a bottommost surface line 138 and a topmost surface line 140, wherein the bottommost surface line 138 is coincident with a straight edge of the bilaterally symmetric, longitudinal cross section of the catheter tip 104 shown in FIG. 3 and the topmost surface line 140 is coincident with a gently sigmoidal edge of the foregoing longitudinal cross section of the catheter tip 104. The taper angle can vary in that the bottommost surface line 138 can be straight, the topmost surface line 140 can be gently sigmoidal, and the surface lines therebetween from the bottommost surface line 138 to the topmost surface line 140 can be increasingly sigmoidal with an entirety of the bottommost surface line 138 having the least taper angle (e.g., about 0.0°) and the topmost surface line 140 having a medial portion achieving the greatest taper angle, which can be less than or equal to about 15.0°, including less than or equal to about 10.0°, such as less than or equal to about 7.5°, for example, less than or equal to about 5.0°.

The catheter tip 104 can but need not be formed of a first, harder polymeric material than the second polymeric material of the catheter tube 102 set forth above. In other words, the first polymeric material can have a first durometer greater than the second durometer of the second polymeric material. The first durometer can be a Shore A durometer of about 70-100, including a Shore A durometer of about 80-100, such as a Shore A durometer of about 90-100, for example, a Shore durometer of about 90-95. Such a first polymeric material can include, but is not limited to, a polytetrafluoroethylene, a polypropylene, or a polyurethane having the first durometer greater than the second durometer of the second polymeric material. For example, the catheter tip 104 can be formed of a polyurethane having the first durometer. Like that set forth above with respect to the catheter tube 102, polyurethanes can also be advantageous for the catheter tip 104. Whether or not the first polymeric material is a polyurethane, the first polymeric material can be configured to be sufficiently pliable in vivo at body temperature to prevent trauma to a blood-vessel lumen of a patient when the RICC 100 is advanced into the blood-vessel lumen per the method of placing the RICC 100 set forth below. However, the first polymeric material can be configured to soften less than the second polymeric material in vivo at body temperature, in a presence of moisture, or both. Indeed, the first polymeric material can be configured to remain sufficiently stiff to prevent the catheter tip 104 from collapsing, buckling, or otherwise appreciably deforming when the RICC 100 is advanced into the blood-vessel lumen of the patient. Additionally, or alternatively, the first polymeric material can be configured to remain sufficiently stiff to prevent the catheter-tip lumen 124 of the catheter tip 104 from collapsing when aspirating through the catheter tip 104.

It should be understood the first durometer of the first polymeric material and the second durometer of the second polymeric material can be on different hardness scales (e.g., Type A or Type D), each of which ranges from 0 to 100. With this understanding, the second durometer of the second polymeric material might not be numerically less than the first durometer of the first polymeric material when the second durometer is less than the first durometer. Indeed, the hardness of the second polymeric material can still be less than the hardness of the first polymeric material as the different hardness scales are designed for characterizing different materials in groups of the materials having a like hardness.

The catheter hub 106 can include one or more catheter-hub lumens corresponding in number to the one or more catheter-tube lumens such as the three catheter-hub lumens set forth above, which correspond in number to the three catheter-tube lumens 118, 120, and 122 also set forth above. When the RICC 100 is a triluminal RICC, the catheter hub 106 can include a catheter-hub portion of the primary lumen 112 of the RICC 100, a catheter-hub portion of the secondary lumen 114 of the RICC 100, and a catheter-hub portion of the tertiary lumen 116 of the RICC 100. The one or more catheter-hub lumens can extend through an entirety of the catheter hub 106 from a proximal end of the catheter hub 106 to a distal end of the catheter hub 106.

The one or more extension legs 108 can respectively include one or more extension-leg lumens corresponding in number to the one or more catheter-hub lumens such as the three extension-leg lumens set forth above, which correspond in number to the three catheter-hub lumens also set forth above. When the RICC 100 is a triluminal RICC, the one or more extension legs 108 can include a primary extension leg including an extension-leg portion of the primary lumen 112 of the RICC 100, a secondary extension leg including an extension-leg portion of the secondary lumen 114 of the RICC 100, and a tertiary extension leg including an extension-leg portion of the tertiary lumen 116 of the RICC 100. Each extension-leg lumen of the one or more extension-leg lumens can extend through an entirety of its corresponding extension leg from a proximal end of the extension leg to a distal end of the extension leg.

Notably, any component of the RICC 100 selected from the catheter tip 104, the catheter tube 102, the catheter hub 106, the one or more extension legs 108, and the one or more extension-leg connectors 110 can include an antimicrobial thereon or therein. In an example, the catheter tube 102 and the catheter tip 104 coupled thereto can include an antimicrobial coating on the abluminal surfaces of the catheter tip 104 and the catheter tube 102. In another example, a pre-extrusion material of the catheter tube 102 can include the antimicrobial admixed therein such that the antimicrobial is incorporated into the catheter tube 102 when extruded, the antimicrobial protecting both the abluminal surface of the catheter tube 102 and the luminal surface of the catheter tube 102 from microbial contamination. Additionally, or alternatively, the polymeric plug 150 of the first polymeric material can include the antimicrobial admixed therein such that the antimicrobial is incorporated into the catheter tip 104 when formed as set forth below, the antimicrobial protecting both the abluminal surface of the catheter tip 104 and the luminal surface of the catheter tip 104 from microbial contamination.

### Methods

Methods include at least methods of forming the catheter tip 104 of the RICC 100 as well as placing such a RICC in a blood-vessel lumen of a patient.

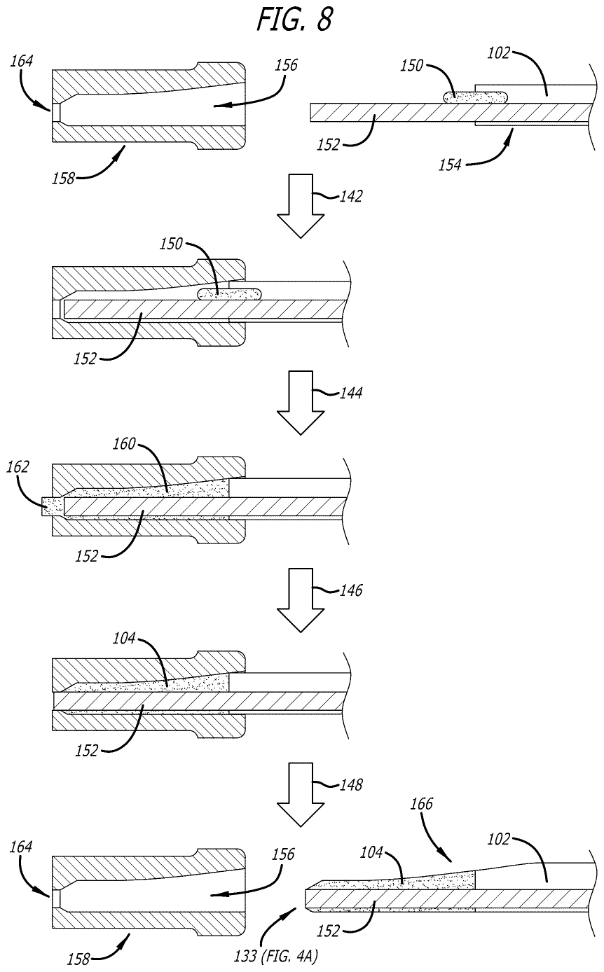

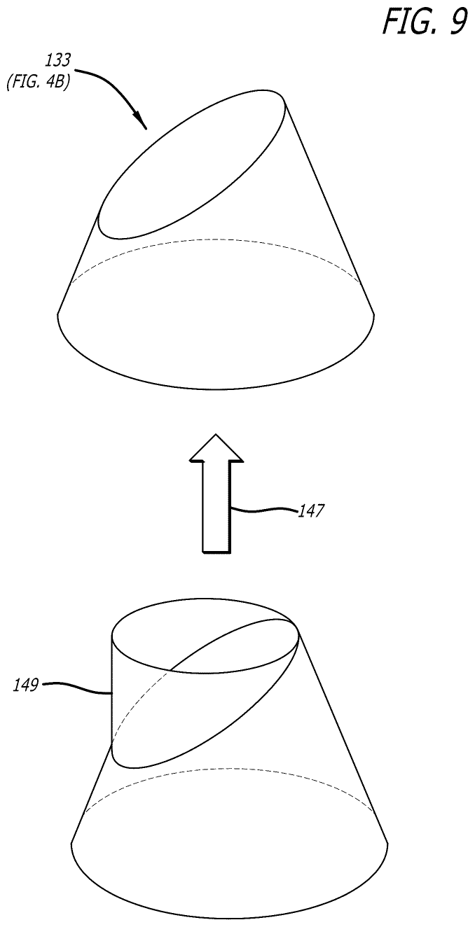

FIGS. 8 and 9 illustrate a method for forming the catheter tip 104 in accordance with some embodiments.

A method for forming the catheter tip 104 of the RICC 100 can include one or more steps selected from a catheter-tube-obtaining step, a polymeric-plug-inserting step, a catheter-tube-mounting step, a workpiece-inserting step 142, a die-heating step 144 in accordance with radiofrequency (“RF”) welding, a mandrel-thrusting step 146, a workpiece-removing step 148, and a mandrel-removing step.

The catheter-tube-obtaining step can include either sourcing or extruding the catheter tube 102 of the second polymeric material, the second polymeric material being softer than the first polymer material of the catheter tip 104 as set forth above. Again, the catheter tube 102 can include the primary catheter-tube lumen 118, the secondary catheter-tube lumen 120, and the tertiary catheter-tube lumen 122 corresponding to catheter-tube portions of the primary lumen 112, the secondary lumen 114, and the tertiary lumen 116 of the RICC 100, respectively.

The polymeric-plug-inserting step can include at least partially inserting a polymeric plug 150 of the first polymeric material into each lumen of the secondary and tertiary catheter-tube lumens 120 and 122 for melting the first polymeric material in the cavity 156 of the tipping die 158 while heating the tipping die 158 in the die-heating step 144 set forth below. That said, the method of forming the catheter tip 104 can alternatively include melting the second polymeric material of the catheter tube 102 in the cavity 156 of the tipping die 158 while heating the tipping die 158 in the die-heating step 144 set forth below. In such embodiments, the polymeric-plug-inserting step is not performed.

The catheter-tube-mounting step can include mounting the catheter tube 102 on a straight mandrel 152 to form a mandrel-mounted catheter tube 154 for a workpiece, optionally, with the polymeric plug 150 of the first polymeric material inserted into each lumen of the secondary and tertiary catheter-tube lumens 120 and 122, as the catheter-tube-mounting step can be performed either before or after the polymeric-plug-inserting step.

The workpiece-inserting step 142 can include inserting the mandrel-mounted catheter tube 154 into a cavity 156 of an tipping die 158. A distal end of the mandrel 152 of the mandrel-mounted catheter tube 154 can remain short of an end of the cavity 156 after the inserting of the mandrel-mounted catheter tube 154 into the cavity 156.

The die-heating step 144 can include heating the tipping die 158 to melt the first polymeric material of the polymeric plug 150 extending from each lumen of the secondary and tertiary catheter-tube lumens 122 and 124 in the cavity 156 of the tipping die 158. During the heating of the tipping die 158, molten polymeric material of the first polymeric material can conform to the cavity 156 around a distal portion of the mandrel 152 to form, in a single piece, a nascent catheter tip 160 coupled to the distal end portion of the catheter tube 102. If, as above, the method of forming the catheter tip 104 alternatively includes melting the second polymeric material of the catheter tube 102 instead of that of the polymeric plug 150, heating the tipping die 158 provides molten polymeric material of the second polymeric material that can conform to the cavity 156 around the distal portion of the mandrel 152 to form, in a single piece, the nascent catheter tip 160 coupled to the distal end portion of the catheter tube 102.

The mandrel-thrusting step 146 can include thrusting the distal end of the mandrel 152 into the end of the cavity 156 of the tipping die 158 when molten exudate 162 of the molten polymeric material exudes through an exit hole 164 in the end of the cavity 156. Excess polymeric material can be cut from a distal end of the nascent catheter tip 160 with the thrusting of the distal end of the mandrel 152 into the end of the cavity 156 to provide a mandrel-mounted tipped catheter tube 166 for the workpiece, a tipped catheter tube thereof including the catheter tip 104 coupled to the distal end portion of the catheter tube 102.

Notably, the bevel 133 of the catheter tip 104 shown in FIG. 4A directly results from the mandrel-thrusting step 146. While the mandrel-thrusting step 146 can be used to cut some of the excess polymeric material from the distal end of the nascent catheter tip 160 en route to the bevel 133 of the catheter tip 104 shown in FIG. 4B, the method of forming such a catheter tip 104 can further include a bevel-cutting step 147 to remove a cylindrical wedge 149 of a remainder of the excess polymeric material as shown in FIG. 9 to form the bevel 133.

The workpiece-removing step 148 can include removing the mandrel-mounted tipped catheter tube 166 from the cavity 156 of the tipping die 158. Notably, a remainder of the polymeric plug 150 in the secondary and tertiary catheter-tube lumens 120 and 122, melted polymeric material of the first polymeric material in the secondary and tertiary catheter-tube lumens 120 and 122, or a combination thereof in the secondary and tertiary catheter-tube lumens 120 and 122 terminate or close off the secondary and tertiary catheter-tube lumens 120 and 122 as infill upon cooling the mandrel-mounted tipped catheter tube 166 or, after the mandrel-removing step set forth below, the tipped catheter tube thereof.

The mandrel-removing step can include removing the mandrel 152 from the mandrel-mounted tipped catheter tube 166 to clear a primary lumen of the tipped catheter tube of the mandrel 152, thereby providing the tipped catheter tube. Notably, cutting, optionally, laser cutting, to remove the cylindrical wedge 149 in the bevel-cutting step 147 can be performed upon partially withdrawing the mandrel 152 from the mandrel-mounted tipped catheter tube 166 such that a distal end of the mandrel 152 is just proximal of the cylindrical wedge 149. That said, the bevel-cutting step 147 can alternatively be performed upon completely removing the mandrel 152 from the mandrel-mounted tipped catheter tube 166.

Due to forming the catheter tip 104 of the RICC 100 in accordance with the foregoing method, the abluminal surface of the tapered portion 136 of the catheter tip 104 can smoothly transition to the abluminal surface of the catheter tube 102 without edges that catch on the skin of the patient when the when the RICC 100 is advanced into the blood-vessel lumen per the method of placing the RICC 100 set forth below.

While not shown, a method of placing the RICC 100 having the catheter tip 104 can include one or more steps selected from a needle-tract-establishing step, an access-guidewire-advancing step, an introducer-needle-withdrawing step, an initial RICC-advancing step including a dilating step or operation performed simultaneously therewith, a guidewire-exchanging step including an access-guidewire-withdrawing step and an initial maneuver-guidewire-advancing step, a subsequent maneuver-guidewire-advancing step, a subsequent RICC-advancing step, and a maneuver-guidewire-withdrawing step.

The needle-tract-establishing step can include establishing a needle tract from an area of skin to a blood-vessel lumen of a patient with an introducer needle.

The access-guidewire-advancing step can include advancing a distal portion of an access guidewire into the blood-vessel lumen through the introducer needle until access to the blood-vessel lumen is secured with the access guidewire.

The introducer-needle-withdrawing step can include withdrawing the introducer needle from the blood-vessel lumen leaving the access guidewire in place in the blood-vessel lumen.

The initial RICC-advancing step can include advancing a distal portion of the RICC 100 over the access guidewire into the blood-vessel lumen until access to the blood-vessel lumen is secured with the catheter tube 102. Neither the skin nor other tissue around the needle tract catches between an outer diameter of the access guidewire and an inner diameter of the catheter tip 104 due to tight control of manufacturing tolerance between the foregoing diameters. As set forth above, the distal portion of the RICC 100 can include the catheter tip 104 of the first polymeric material having the beveled section 132, the straight portion 134, and the tapered portion 136 coupled to the distal end portion of the catheter tube 102 of the second polymeric material. The first polymeric material of the catheter tip 104 can be configured to remain sufficiently stiff to prevent the catheter tip 104 from collapsing, buckling, or otherwise appreciably deforming while advancing the distal portion of the RICC 100 over the access guidewire into the blood-vessel lumen in accordance with the initial RICC-advancing step.

The dilating step or operation can include dilating the tissue around the needle tract leading to the blood-vessel lumen while advancing the distal portion of the RICC 100 over the access guidewire into the blood-vessel lumen in accordance with the initial RICC-advancing step. As such, the dilating step or operation is part of the initial RICC-advancing step—not a separate step or operation therefrom like that needed with the Seldinger technique. As set forth above, the beveled section 132 of the catheter tip 104 includes the bevel 133 configured for immediately dilating the tissue around the needle tract from the size commensurate with the outer diameter of the needle shaft of the introducer needle to the size commensurate with the outer diameter of the distalmost portion of the main section of the catheter tip 104, specifically, the outer diameter of the straight portion 134 of the catheter tip 104. The tapered portion 136 of the catheter tip 104 has the non-uniform taper over the outer diameter of the tapered portion 136 configured for further dilating the tissue from the size commensurate with the proximalmost portion of the beveled section 132 or the outer diameter of the straight portion 134 of the catheter tip 104 to the size commensurate with the outer diameter of the catheter tube 102.

The exchanging step can include exchanging the access guidewire with a maneuver guidewire, which, in turn, can include the access-guidewire-withdrawing step and the initial maneuver-guidewire-advancing step. The access-guidewire-withdrawing step can include withdrawing the access guidewire from the blood-vessel lumen leaving the catheter tube 102 in place in the blood-vessel lumen. The initial maneuver-guidewire-advancing step can include advancing a distal portion of the maneuver guidewire into the blood-vessel lumen.

The subsequent maneuver-guidewire-advancing step can include advancing the distal portion of the maneuver guidewire farther into the blood-vessel lumen to a lower ⅓ of a superior vena cava (“SVC”) of a heart of the patient.

The subsequent RICC-advancing step can include advancing the distal portion of the RICC 100 farther into the blood-vessel lumen over the maneuver guidewire to the lower ⅓ of the SVC. Notably, the first polymeric material is also sufficiently pliable to prevent trauma to the blood-vessel lumen while advancing the distal portion of the RICC 100 over the maneuver guidewire farther into the blood-vessel lumen in accordance with the subsequent RICC-advancing step.

The maneuver-guidewire-withdrawing step can include withdrawing the maneuver guidewire leaving the catheter tube 102 in place in the lower ⅓ of the SVC.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. A rapidly insertable central catheter (“RICC”), comprising:
a catheter tube including a catheter-tube portion of a primary lumen of the RICC;
a single-piece catheter tip coupled to a distal end portion of the catheter tube, the catheter tip including:
a beveled section including a bevel approximating an obliquely truncated cone;
a main section proximal of the beveled section; and
a catheter-tip portion of the primary lumen of the RICC;
a catheter hub coupled to a proximal end portion of the catheter tube; and
one or more extension legs, each extension leg of the one or more extension legs coupled to the catheter hub by a distal end portion thereof.

2. The RICC of claim 1, wherein the beveled section is configured for dilating tissue around a needle tract from a size commensurate with an outer diameter of a needle shaft of an introducer needle to a size commensurate with an outer diameter of a distalmost portion of the main section of the catheter tip.

3. The RICC of claim 1, wherein the catheter-tip portion of the primary lumen of the RICC is offset with respect to a center of a transverse cross section of the main section of the catheter tip.

4. The RICC of claim 3, wherein the bevel is coaxial with respect to the catheter-tip portion of the primary lumen of the RICC, the bevel having a circular perimeter around its distal opening concentric with an inner diameter of the catheter-tip portion of the primary lumen of the RICC.

5. The RICC of claim 4, wherein the bevel has an elliptical base oblique to its distal opening.

6. The RICC of claim 1, wherein the main section of the catheter tip includes a straight portion and a tapered portion proximal of the straight portion, the straight portion of the main section having a consistent outer diameter, and the tapered portion of the main section having a non-uniform taper configured for dilating tissue around a needle tract from a size commensurate with an outer diameter of a proximalmost portion of the beveled section to a size commensurate with an outer diameter of the distal end of the catheter tube.

7. The RICC of claim 6, wherein the bevel is coaxial with respect to the straight portion of the main section of the catheter tip, the bevel having a circular perimeter around its base concentric with the outer diameter of the straight portion of the main section.

8. The RICC of claim 7, wherein the bevel has an elliptical distal opening oblique to its base.

9. The RICC of claim 6, wherein each successive transverse cross section of the tapered portion of the main section of the catheter tip approximates a larger eccentric annulus in a proximal direction and a smaller eccentric annulus in a distal direction along the tapered portion of the main section, each eccentric annulus of the foregoing larger and smaller eccentric annuli defined by an outer diameter of the tapered portion of the main section and an inner diameter of the catheter-tube portion of the primary lumen of the RICC, thereby providing the non-uniform taper of the tapered portion of the main section of the catheter tip.

10. The RICC of claim 1, wherein the catheter tip is of a first polymeric material and the catheter tube is of a second, softer polymeric material.

11. The RICC of claim 10, wherein the first polymeric material is sufficiently stiff to prevent the catheter tip from collapsing, buckling, or otherwise appreciably deforming when a distal portion of the RICC is advanced into a blood-vessel lumen of a patient, the first polymeric material also being sufficiently pliable to prevent trauma to the blood-vessel lumen when the distal portion of the RICC is advanced farther into the blood-vessel lumen.

12. The RICC of claim 10, wherein the RICC includes a set of three lumens including the primary lumen, a secondary lumen, and a tertiary lumen, the secondary and tertiary lumens terminating in the distal end portion of the catheter tube by at least some infill of melted polymeric material of the first polymeric material.

13. The RICC of claim 12, wherein the primary lumen has a primary-lumen aperture in a distal opening of the catheter tip, the secondary lumen has a secondary-lumen aperture in a side of the distal end portion of the catheter tube, and the tertiary lumen has a tertiary-lumen aperture in the side of the distal end portion of the catheter tube but proximal of the secondary-lumen aperture.

14. A catheter tip, comprising:
a beveled section including a bevel approximating an obliquely truncated cone;
a main section proximal of the beveled section; and
a lumen through an entirety of the catheter tip.

15. The catheter tip of claim 14, wherein the lumen is offset with respect to a center of a transverse cross section of the main section of the catheter tip.

16. The catheter tip of claim 15, wherein the bevel is coaxial with respect to the lumen, the bevel having a circular perimeter around its distal opening concentric with an inner diameter of the lumen.

17. The catheter tip of claim 16, wherein the bevel has an elliptical base oblique to its distal opening.

18. The catheter tip of claim 14, wherein the main section of the catheter tip includes a straight portion and a tapered portion proximal of the straight portion, the straight portion of the main section having a consistent outer diameter, and the tapered portion of the main section having a non-uniform taper.

19. The catheter tip of claim 18, wherein the bevel is coaxial with respect to the straight portion of the main section of the catheter tip, the bevel having a circular perimeter around its base concentric with the outer diameter of the straight portion of the main section.

20. The catheter tip of claim 19, wherein the bevel has an elliptical distal opening oblique to its base.

21. The catheter tip of claim 18, wherein each successive transverse cross section of the tapered portion of the main section of the catheter tip approximates a larger eccentric annulus in a proximal direction and a smaller eccentric annulus in a distal direction along the tapered portion of the main section, each eccentric annulus of the foregoing larger and smaller eccentric annuli defined by an outer diameter of the tapered portion of the main section and an inner diameter of the lumen, thereby providing the non-uniform taper of the tapered portion of the main section of the catheter tip.
